(19) United States
(12) Patent Application Publication
(10) Pub. No.: US 2024/0105327 A1
(43) Pub. Date: Mar. 28, 2024

(54) SYSTEM AND METHOD OF LIMITED MOBILITY TRACKING

(71) Applicant: Zinfanite Technologies, Inc., Torrance, CA (US)

(72) Inventors: Jana Mahen Fernando, Torrance, CA (US); Ali Reza Kharrazi, Rancho Cucamonga, CA (US)

(21) Appl. No.: 17/950,913

(22) Filed: Sep. 22, 2022

(51) Int. Cl. G16H 40/63; G06F 1/16

(52) U.S. Cl. CPC G16H 40/63; G06F 1/163

(57) ABSTRACT

An electronic device is disclosed. The electronic device includes an accelerometer configured to detect movement of a user of the electronic device; a user interface configured to receive user settings comprising a type of movements, a level of sensitivity of the accelerometer, and a placement of the electronic device on the user; a non-transitory memory configured to store instructions; and a processor connected to the accelerometer, user interface, and non-transitory memory, the processor configured to execute instructions stored in the non-transitory memory to: capture motion data from the accelerometer based on the type of movements, the level of sensitivity of the accelerometer, and the placement of the electronic device on the user.

### FIELD

This relates generally to a system and method of tracking motion, and more particularly, to fitness tracking device for people with limited mobility.

### BACKGROUND

There are numerous fitness tracking devices in the world. Existing fitness tracking devices track movement without the ability to track and identify the exact type of exercise being performed by the user especially when the movement by the user does not fit the profile of a routine exercise. For example, people who are fully mobile use fitness tracking devices that track the number of steps they walk, which may be adequate to estimate the user's overall exercise for that day. However, for individuals with limited mobility or those undergoing physical therapy, there is a need to track unique movements and exercises they are doing on a regular basis.

### SUMMARY

A novel fitness tracking device is desired by users with limited mobility to maintain their physical health and allow doctors to monitor their physical conditions such as mobility and/or progress in therapy. A person with limited mobility may not be able to walk or trigger typical fitness tracking devices, because their motions may not follow a regular pattern recognizable by the conventional fitness tracking devices. Their motion may be too soft for the conventional fitness trackers to sense. In some cases, even if a conventional fitness tracker can track the motion of a user with limited mobility, it has difficulties in finding a movement pattern, recognizing the movement, and/or extracting meaningful information from the movement data. Some users may not have limbs or be able to wear the conventional fitness trackers as intended and those devices would therefore not be tracking user movement correctly.

To solve these issues, various embodiments of a fitness tracking device are disclosed below. Embodiments of the disclosed device can track a range of motions. In addition, the device can be tuned in terms of sensitivity and the user can select which motions to track or program the device to recognize certain motions. In one embodiment, the device can be a medallion that can be worn around the chest, arm, leg, wrist or ankle.

### BRIEF DESCRIPTION OF THE DRAWINGS

FIG. 1 illustrates the exemplary components of a system for capturing and analyzing user movements and motions, according to an embodiment of the disclosure.

FIG. 2 illustrates the exemplary components of a wearable, according to an embodiment of the disclosure.

FIGS. 3a-3c illustrates exemplary screen shots from a motion tracking program/app running on the user device 102 of FIG. 2, according to an embodiment of the disclosure.

FIGS. 4a-4q show different exemplary motions and movements that a user would be able to perform and be tracked by the medallion (or other embodiments of the wearable device 102 of FIG. 1).

### DETAILED DESCRIPTION OF THE PREFERRED EMBODIMENTS

In the following description of preferred embodiments, reference is made to the accompanying drawings which form a part hereof, and in which it is shown by way of illustration specific embodiments, which can be practiced. It is to be understood that other embodiments can be used and structural changes can be made without departing from the scope of the embodiments of this disclosure.

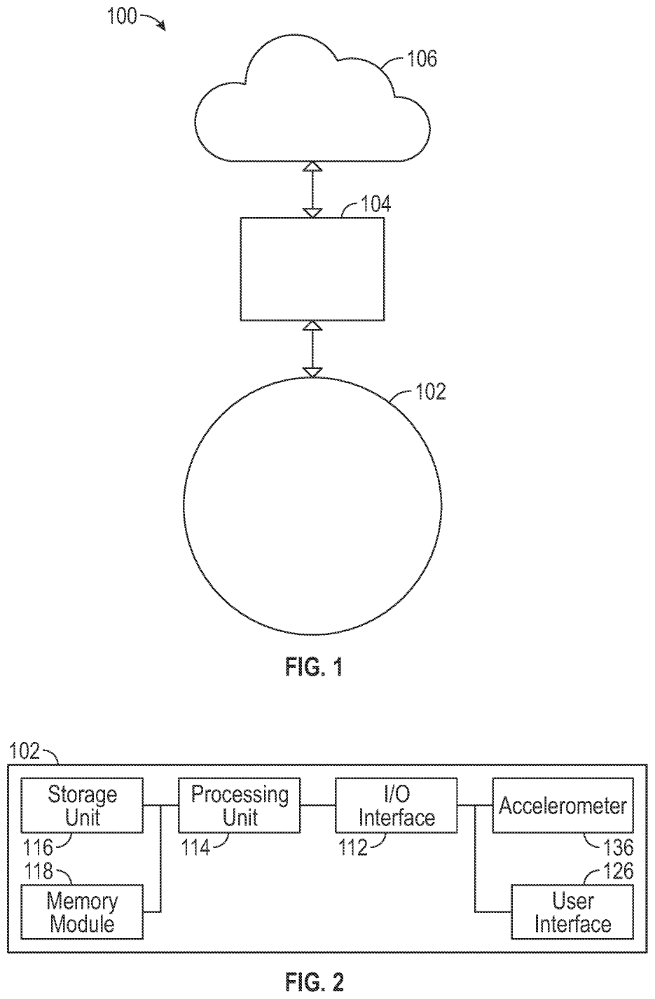

FIG. 1 illustrates the exemplary components of a system for capturing and analyzing user movements and motions, according to an embodiment of the disclosure. The system 100 includes a wearable device 102 that is designed to be worn in different ways by different groups of targeted users. The wearable device 102 can track a range of movements and motions that a person might perform. The wearable device 102 can be connected to another device such as a user's mobile phone 104 via Bluetooth or any other types of networks. The mobile phone 104 can be connected via WiFi or a cellular network or any other suitable network to a cloud server 106.

FIG. 2 illustrates the exemplary components of a wearable device 102 of FIG. 1, according to an embodiment of the disclosure. The wearable device 102 can include, among other things, an I/O interface 112, a processing unit 114, a storage unit 116, a memory module 118, user interface 126, and an accelerometer 136.

I/O interface 112 may also be configured for two-way communication with other components of the user device 102, such as user interface 126 and accelerometer 136. I/O interface 122 may also send and receive data to and from devices such as mobile phone 104 of FIG. 1 (not shown in FIG. 1).

Processing unit 114 may be configured to receive signals and process the signals to determine a plurality of conditions of the operation of user device 102. Processing unit 114 may also be configured to generate and transmit command signals, via I/O interface 112, to actuate components such as accelerometer 136 and user interface 126.

Storage unit 116 and/or memory module 118 may be configured to store one or more computer programs that may be executed by processing unit 114 to perform functions of the user device 102. For example, storage unit 116 and/or memory module 118 may be configured to store a motion tracking program (or app), which can allow a user to calibrate, via the user interface 126, the accelerometer 136 for detecting certain types of movements. In one embodiment, the calibration of the accelerometer can be done automatically by the program/app without requiring any user input via the user interface 126. This can be achieved by analyzing accelerometer data and automatically calibrating the accelerometer based on the data. For example, if the accelerometer data is insufficient to generate a profile of the user movement, the accelerometer can be calibrated to increase its sensitivity. In contrast, if the accelerometer data has too much noise, the accelerometer can be calibrated to be less sensitive.

Storage unit 116 and memory 118 can be non-transitory computer-readable medium storing instructions which, when executed, cause one or more processors to perform the method, as discussed below. The computer-readable medium can include volatile or non-volatile, magnetic, semiconductor, tape, optical, removable, non-removable, or other types of computer-readable medium or computer-readable storage devices. The computer-readable medium can have computer instructions stored thereon, as disclosed. In some embodiments, the computer-readable medium may be a disc or a flash drive having the computer instructions stored thereon.

It should be understood that the wearable device 102 of FIG. 2 can include additional components that are not shown in FIG. 2 and that some of the components shown in FIG. 2 may be optional in certain embodiments.

The mobile phone 104 of FIG. 1 can include similar component as the wearable device 102 of FIG. 2. In some embodiments, the user calibration of the accelerometer can be performed on the mobile phone 104 instead of wearable device 102. Indeed, some or all of the data processing required in these embodiments can be performed by either the wearable device 102 or the mobile phone 104 or a combination thereof.

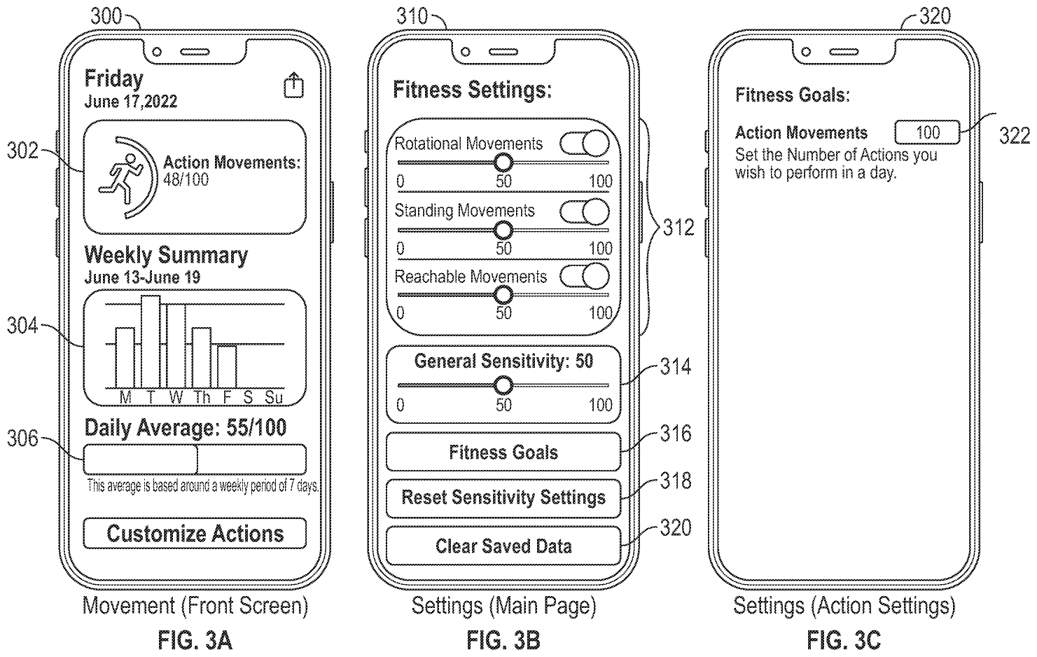

FIGS. 3a-3c illustrates exemplary screen shots from a motion tracking program/app running on the user device 102 or the mobile phone 104 of FIG. 1, according to an embodiment of the disclosure. FIG. 3a is an exemplary “Summary” screen 300 of the program showing, for example, the action movements being tracked for the day 302, a weekly summary graph 304 showing the daily amount of movement/exercise by the user for the entire week, and a daily average 306 of the amount of movement/exercise for the week.

FIG. 3b illustrates an exemplary “Settings” screen 310 of the motion tracking program/app. The user can select the type of movement(s) (e.g., rotational, standing, reachable) 312 to be captured by the wearable device. In one embodiment, the program/app can allow the user to input a customized movement by switching the program/app to a motion capturing mode and record the movement of the user using the wearable device. The program/app can ask the user to perform the customized movement multiple times to accurately record the movement. Once the customized movement is recorded by the program/app, it can be made available via the “Settings” screen 310 for user selection.

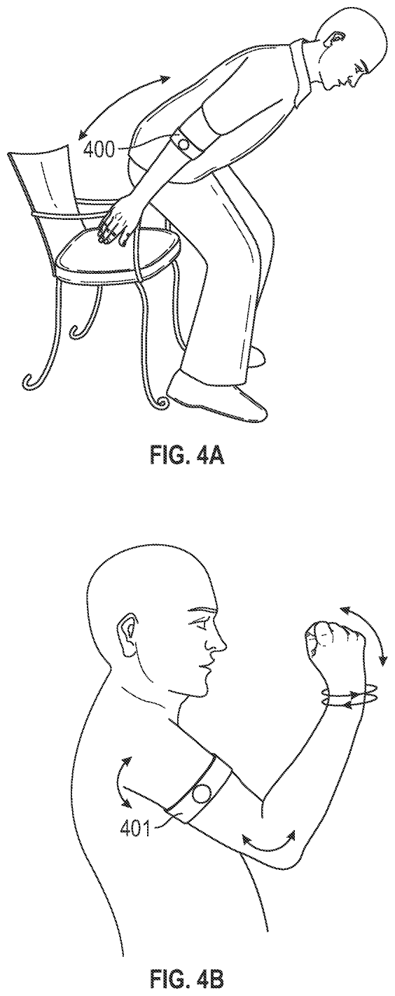

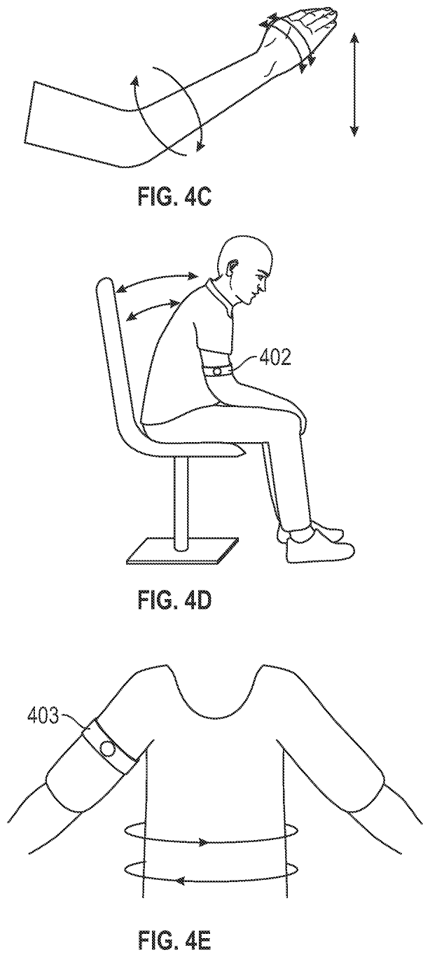

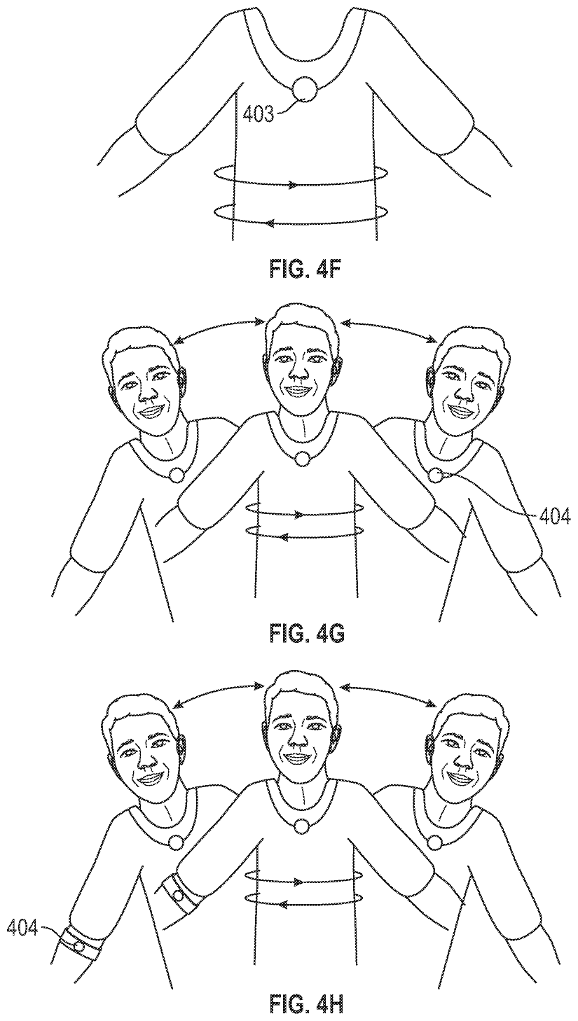

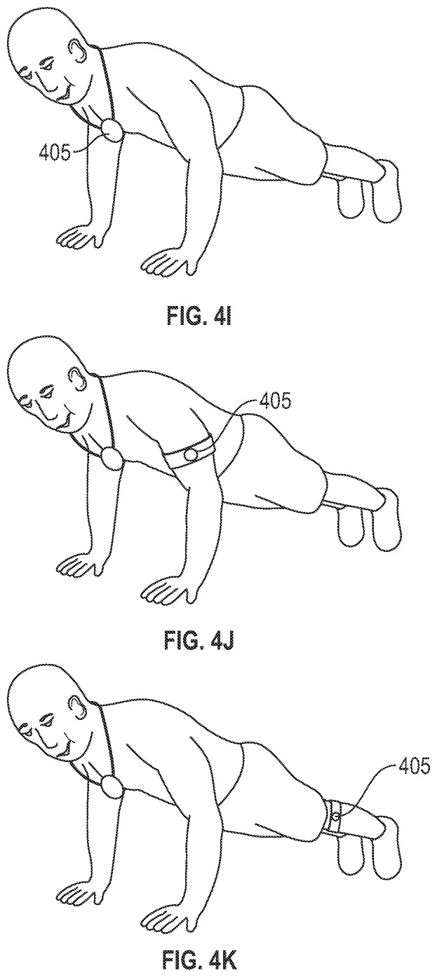

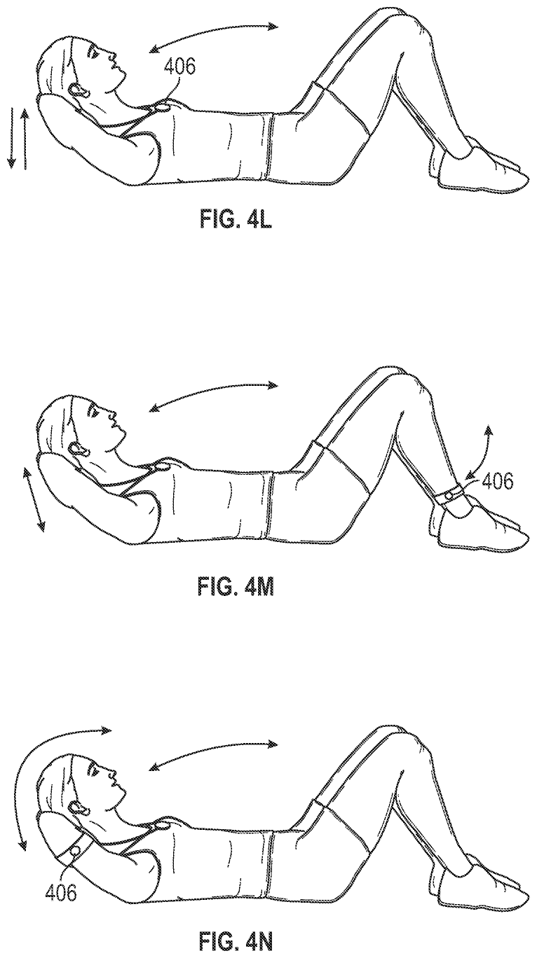

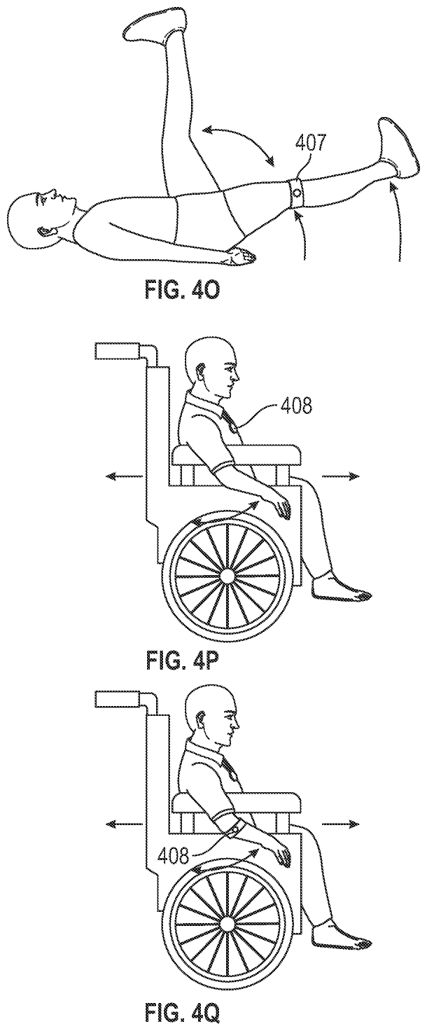

After the user selects the type of movement(s) on the “Settings” screen 310, the program/app can provide instructions on how the user should wear the wearable device. For example, if the user selects a “getting up from a chair” movement (as shown in FIG. 4a), the program/app can instruct the user to wear the wearable device around the arm. This instruction can be displayed on a screen of the program/app.

The user can also set the general sensitivity 314 of the selected movement. Additionally or alternatively, the user can set short term or long-term fitness goals 316, reset sensitivity settings 318, and/or clear saved data 320 by interacting with the menu items displayed on the Setting screen 310. The processor of the wearable device can tune the components including the accelerometer in response to the settings entered by the user so the wearable device can be optimized for detecting and capturing information from the particular movement(s) selected by the user.

FIG. 3c illustrates an exemplary “Fitness Goals” screen 320 of the motion tracking program/app. The user can set, for example, the number of actions he/she wishes to perform in a day (or any other time frame) 322. The program/app can use this information to provide feedback to the user with regard to if he/she is meeting his/her goals.

Additional or alternative information can be provided on each of these exemplary screens of FIGS. 3a-3c. Additional or alternative screens can be included in the motion tracking program/app.

In some embodiments, the program/app can capture and record where the wearable device is worn on the user and automatically adjust the settings such as sensitivity of the accelerometer based on how the device is worn. For example, when the device is worn on the wrist or ankle, the sensitivity of the accelerometer can be automatically adjusted to less sensitive due to the frequent movements of the wrist or ankle during most exercises. In contrast, when the device is worn on the chest or around the thigh, the sensitivity of the accelerometer can be automatically increased. In some embodiments, the sensitivity level can automatically be adjusted based on a number of factors including but not limited to how the device is worn and the type of motion to be tracked. The sensitivity level can automatically be restricted to a range based on how the device is worn, the type of motion to be tracked, or both. The type of motion to be tracked can be limited based on how the device is worn. For example, if the device is worn on an ankle, the user interface will not permit the user to select movements for an arm exercise. Similarly, if a user selects movements for an arm exercise, the app can instruct the user to put the device on his/her arm.

Referring back to FIG. 1, the wearable device 102 can be a medallion in one embodiment of the disclosure. The medallion also includes different accessories that allow it to be worn near the chest, arm, wrist, thigh, ankle, and/or other parts of a user's body depending on the movements to be tracked.

FIGS. 4a-4q show different exemplary motions and movements that a user would be able to perform and be tracked by the medallion (or other embodiments of the wearable device 102 of FIG. 1). FIG. 4a illustrates a user getting up from a chair. The medallion can be worn on the chest (or arm). In either case, the medallion 400 would be able to profile that motion and record it as an activity. In order to perform this activity tracking, the accelerometer on the medallion 400 can record raw data, which is then transferred to a mobile phone (e.g., device 104 of FIG. 1) and/or perhaps to a cloud computing system (e.g., cloud server 106 of FIG. 1) that can provide an accurate profile the tracked motion.

FIG. 4b illustrates the medallion 401 worn around a user's upper arm. In this embodiment, the motion being tracked can be any movement of the arm such as, but not limited to, lifting the arm up or to the side and/or rotating the arm. The medallion 401 can also track the motion of stretching the arms outwards and bring them outwards and inwards, as shown in FIG. 4c.

FIG. 4d illustrates the medallion 402 worn around a user's upper arm. In this embodiment, the motion being tracked by the medallion is the rocking backwards and forwards of the user's upper body.

FIGS. 4e and 4f illustrate the medallion 403 worn around a user's upper arm and on the chest, respectively, to track a twisting motion of the torso.

FIGS. 4g and 4h illustrate the medallion 404 worn around a user's upper arm and on the chest, respectively, to track the user's torso moving from side to side.

FIGS. 4i-4k illustrate the medallion 405 being worn on the chest, around the upper arm, and around the lower leg, respectively, to track user's motion when performing a push-up.

FIGS. 4l-4n illustrate the medallion 406 worn on the chest, around the ankle, and around the upper arm, respectively, to track user's motion when performing a sit-up.

FIG. 4o illustrates the medallion 407 worn around the user's thigh to track the motion of raising legs.

FIGS. 4p and 4q illustrate the medallion 408 worn on the user's chest and around the lower arm, respectively, to track user's motion of rolling a wheelchair forwards and backwards while the user is sitting in the wheelchair.

It should be understood that the examples provided above and illustrated in FIGS. 4a-4q are not limiting. Embodiments of the wearable device can be designed to be worn in other different ways and capable of capture and recognize different motions. For example, the wearable device can be designed to be worn as part of a headband for capturing a wearer's head movements.

Referring again to FIG. 1, motion data captured by the wearable device 102 can be stored in the wearable device and/or transmitted via a network (e.g., Bluetooth) to the mobile phone 104 and/or the cloud server 106 for processing.

All of the methods and tasks described herein may be performed and fully automated by a computer system. The computer system may, in some cases, include multiple distinct computers or computing devices (e.g., physical servers, workstations, storage arrays, cloud computing resources, and mobile devices, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Although embodiments of this disclosure have been fully described with reference to the accompanying drawings, it is to be noted that various changes and modifications will become apparent to those skilled in the art. Such changes and modifications are to be understood as being included within the scope of embodiments of this disclosure as defined by the appended claims.

What is claimed is:

1. An electronic device comprising:
an accelerometer configured to detect movement of a user of the electronic device;
a user interface configured to receive user settings comprising a type of movements, a level of sensitivity of the accelerometer, and a placement of the electronic device on the user;
a non-transitory memory configured to store instructions; and
a processor connected to the accelerometer, user interface, and non-transitory memory, the processor configured to execute instructions stored in the non-transitory memory to:
capture motion data from the accelerometer based on the type of movements, the level of sensitivity of the accelerometer, and the placement of the electronic device on the user.

2. The electronic device of claim 1, wherein the placement of the electronic device comprises one of the user's chest, upper arm, ankle, wrist, thigh, and shoulder.

3. The electronic device of claim 1, wherein the type of movements comprises one or more of rotational movements, standing movements, and reachable movements.

4. The electronic device of claim 1, wherein the user settings further comprise one or more fitness goals.

5. The electronic device of claim 4, wherein the one or more fitness goals comprise a number of actions the user wishes to perform in a day.

6. The electronic device of claim 5, wherein the user interface is further configured to display user's progress towards the one or more fitness goals.

7. The electronic device of claim 1, wherein the processor is configured to automatically adjust the level of sensitivity of the accelerometer based on the type of movements selected by the user.

8. The electronic device of claim 1, wherein the processor is configured to automatically adjust the level of sensitivity of the accelerometer based on the placement of the electronic device on the user.

9. The electronic device of claim 1, wherein the processor is configured to automatically adjust the level of sensitivity of the accelerometer based on both the type of movements selected by the user and the placement of the electronic device on the user.

10. The electronic device of claim 1, further comprising an I/O interface configured to connect the electronic device to a mobile device or a cloud server.

11. The electronic device of claim 10, wherein the processor is configured to transmit motion data to the mobile device or cloud server for processing.

12. The electronic device of claim 1, wherein the processor is configured to automatically restrict the type of movements that can be selected by the user in response to how the electronic device is worn by the user.

13. The electronic device of claim 1, wherein the processor is configured to provide instructions on how to wear the electronic device in response to a user selection of the type of the movements.

14. The electronic device of claim 1, wherein the processor is further configured to:
analyze the motion data; and
create, based on the motion data, a new type of movement for user selection via the user interface.

15. The electronic device of claim 1, wherein the processor is further configured to calibrate the level of sensitivity of the accelerometer in response to captured motion data.
